(19) United States
(12) Patent Application Publication
(10) Pub. No.: US 2009/0038021 A1
(43) Pub. Date: Feb. 5, 2009

(54) Novel clock gene and application of the same

(75) Inventor: Tomoyuki Hisa, Osaka (JP)

Correspondence Address: HAMRE, SCHUMANN, MUELLER & LARSON, P.C., P.O. BOX 2902, MINNEAPOLIS, MN, 55402-0902, US

(73) Assignees: Tomoyuki Hisa, Izumisano-shi, Osaka (JP); Masazumi Eriguchi, Shinagawa-ku, Tokyo (JP)

(21) Appl. No.: 11/921,334

(22) PCT Filed: Jun. 2, 2006
(86) PCT No.: PCT/JP2006/311119

§ 371 Date: Feb. 11, 2008

(30) Foreign Application Priority Data
Jun. 2, 2005 (JP) 2005-162943

(51) Int. Cl. A01K 67/00; C07H 21/04; C12P 19/34

(52) U.S. Cl. 800/9; 536/23.1; 536/24.33; 435/91.2; 536/24.31; 435/375

(57) ABSTRACT

It is intended to provide a novel gene encoding a new protein which interacts with BMAL2 protein. Namely, a novel gene comprising any one of the following DNAs (a) to (e): (a) a DNA comprising any one of the base sequences represented by SEQ ID NOS: 1 to 4; (b) a DNA that comprises a base sequence derived from the base sequence of the DNA (a) by deletion, substitution or addition of one to several bases and encodes a protein interacting with BMAL2 protein; (c) a DNA that comprises a base sequence derived from the base sequence of the DNA (a) by deletion, substitution or addition of one to several bases and is hybridizable with the DNA (a) under stringent conditions; (d) a DNA that comprises a base sequence derived from the base sequence of the DNA (a) by deletion, substitution or addition of one to several bases and has a homology of 90% or higher with the DNA (a); and (e) a DNA comprising a base sequence that is complementary to any one of the DNAs (a) to (d).

### TECHNICAL FIELD

The present invention relates to a novel gene for encoding a new protein to interact with a BMAL2 protein encoded by a Bmal2 gene as a clock gene. Furthermore, the present invention relates to a primer of the gene and a probe, a method using thereof for detecting the novel gene, a marker gene for diagnosing a biological rhythm disturbance, and a biological rhythm disturbance model animal.

### BACKGROUND ART

It has been known that living organisms act by sensing the time, and that the rhythms are of circadian (one day), circatrigintan (one month), circannual (one year) and the like. Such a biological clock indicates, in general, a circadian rhythm as a vital phenomenon that fluctuates in a daily (24 hours) cycle. A human has a cycle of about 25 hours. For a rat, it is about 24.5 hours, and for a mouse, about 23.5 hours. Though there are some differences among the species, the cycle is approximately 24 hours and considered as being reserved as genes in many species.

For the genes relating to the function of the biological clocks, per (period) gene, tim (timeless) gene, Clk (dock) gene, Bmal (brain and muscle Arnt-like) 1 gene, and cyc gene derived from a fly as a homologue of the Bmal1 gene derived from a mammal are known, for example. Regarding proteins encoded by these genes, for example, it has been known that the partner for a CLOCK protein is a BMAL1 protein, and that these proteins activate transcription of the per gene.

These genes are reserved among various species. For example, the cyc gene has been reserved among the fly and the mouse. Furthermore, several kinds of sub-types have been discovered respectively for the genes of per, tim, cyc (=Bmal1) and clk, and four gene families are formed, for example, per, mPer1 (mouse Per1), mPer2, mPer3, tim, mTim, bmal1, Bmal1, and Bmal2 (see Patent document 1), clk and Clk. Moreover, dbt (double-time) gene family (dbt), cry (cyptochrome) gene family (cry, mCry1, mCry2) and the like have been reported. It is also reported that the Bmal2 gene is identical to the Arnt.

Conventionally, the position of the circadian clock has been studied by a primitive method of destroying various sites of the body. Since the circadian rhythm for action disappears when the suprachiasmatic nucleus is destroyed, this site has been considered as the center of the biological clock. Recently however, it is reported newly that the Bmal2 gene is expressed not only in the brain but in the blood vessel (see Non-patent document 1). According to another report, circadian variations of rPer1 (rat Peroid1) and rPer2 are observed by providing a 50% serum irritation to the fibroblast in vitro (see Non-patent document 2).

Patent document 1: JP 2002-238567 A
Non-patent document 1: Circulation 104, 1746-1748, 2001
Non-patent document 2: Cell 93, 929-937, 1998
Non-patent document 3: JBC 275, 36847-36851, 2000

### DISCLOSURE OF INVENTION

### Problem to be Solved by the Invention

For the purpose of avoiding problems caused by abnormalities in the biological rhythm (such as biological rhythm disturbance), the inventors took notice of the Bmal2 gene exhibiting a peripheral expression. The Bmal2 gene belonging to the Bmal1 family is called also a Clif (cycle-like factor) gene (see Non-patent document 3). This is because, as this gene is the Arnt gene itself, functions of the periphery gene are considered to exist certainly. By clarifying the protein that interacts with the BMAL2 protein encoded by the Bmal2 gene, the molecular mechanism of the biological clock can be clarified further.

Therefore, with the foregoing in mind, it is an object of the present invention to provide a novel clock gene encoding novel protein that interacts with a BMAL2 protein. Another object of the present invention is to provide a primer and a probe that can be used for detecting the gene, a method of gene assay using the primer and the probe, a marker gene for diagnosing a biological rhythm disturbance, and a biological rhythm disturbance model animal.

### Means for Solving Problem

For achieving the above-mentioned objects, the novel clock gene of the present invention is a novel gene comprising any one of the following DNAs (a) to (e):
(a) a DNA comprising any one of the base sequences represented by SEQ ID NOS: 1 to 4;
(b) a DNA that comprises a base sequence derived from the base sequence of the DNA (a) by deletion, substitution or addition of one to several bases and encodes a protein interacting with BMAL2 protein;
(c) a DNA that comprises a base sequence derived from the base sequence of the DNA (a) by deletion, substitution or addition of one to several bases and is hybridizable with the DNA (a) under stringent conditions;
(d) a DNA that comprises a base sequence derived from the base sequence of the DNA (a) by deletion, substitution or addition of one to several bases and has a homology of 90% or higher with the DNA (a); and
(e) a DNA comprising a base sequence that is complementary to any one of the DNAs (a) to (d).

The primer of the present invention is a primer for amplifying a polynucleotide comprising the novel gene of the present invention or the partial sequence, and the polynucleotide comprises a partial sequence of the novel gene of the present invention. The probe of the present invention is a probe for hybridizing with the novel gene of the present invention, and the probe is a polynucleotide comprising the novel gene of the present invention or the partial sequence.

The method of the present invention for analyzing a novel gene includes the following two kinds of assays. That is, the first assay includes a step of performing PCR with respect to a test sample by using the primer of the present invention and a step of investigating the existence of the novel gene depending on the existence of a PCR amplified fragment. The second assay includes a step of incubating a test sample and the probe of the present invention, and a step of investigating the existence of the novel gene by detecting hybridization of the test sample and the probe.

The diagnostic marker gene of the present invention is used for diagnosing a biological rhythm disturbance. The gene is the novel gene of the present invention, and the model animal of the present invention is a biological rhythm disturbance model animal in which the novel gene of the present invention is inactivated or overexpressed.

An antibody of the present invention is a polyclonal antibody or a monoclonal antibody with respect to a protein encoded by the novel gene of the present invention or the partial sequence.

A siRNA of the present invention is a double strand siRNA of 21 bases comprising 19 base pairs and 2-base 3′ overhang, wherein the sequences of the 19 base pairs are any of the SEQ ID NOS: 26 to 28 of the Sequence Listing.

### EFFECTS OF THE INVENTION

As described below, the inventors have identified the novel gene (Erih gene) of the present invention, which encodes a novel protein that interacts with the BMAL2 protein through a two-hybrid assay. In the present specification, the protein encoded by the novel Erih gene of the present invention is called “novel ERIH protein”.

As mentioned above, it has been clarified that the novel ERIH protein encoded by the novel Erih gene of the present invention is associated with the BMAL2 protein on the basis of the affinity. From this fact, it is suggested that the ERIH protein controls the transcriptive activity of the BMAL2 protein through the interaction with the BMAL2 protein. Therefore, by using the novel gene of the present invention, for example, the molecular mechanism of the biological clock relating to the association between the BMAL2 protein and the ERIH protein can be clarified further. In addition to that, by detecting the novel Erih gene of the present invention with use of the primer and the probe of the present invention, for example, a genetic biological rhythm disturbance can be diagnosed. Further, since abnormality in the biological rhythm affects adversely the body, the diagnostic marker gene and the model mouse of the present invention are useful for the diagnosis and the clarification of the mechanism.

### BRIEF DESCRIPTION OF DRAWINGS

FIG. 1 includes a table and a graph referring to an example of the present invention and indicating relative values of hErih1 gene expression levels for various organs of a human.

FIG. 2 includes a table and a graph referring to an example of the present invention and indicating relative values of hErih2 gene expression levels for various organs of a human.

FIG. 3 includes a table and a graph referring to an example of the present invention and indicating relative values of Bmal2 gene expression levels for various organs of a human.

FIG. 4 includes a table and a graph referring to another example of the present invention and indicating relative changes of a hErih1 gene using RNAi in a vascular endothelial cell (HPAEC).

FIG. 5 includes a table and a graph referring to another example of the present invention and indicating relative changes of a hErih2 gene using RNAi in a vascular endothelial cell (HPAEC).

FIG. 6 includes a table and a graph referring to another example of the present invention and indicating relative changes of a Bmal2 gene using RNAi in a vascular endothelial cell (HPAEC).

FIG. 7 includes a table and a graph referring to another example of the present invention and indicating relative changes of a hErih2 gene using RNAi in a bronchial/tracheal epithelial cell (NHBE).

FIG. 8 includes a table and a graph referring to another example of the present invention and indicating relative changes of a Bmal2 gene using RNAi in a bronchial/tracheal epithelial cell (NHBE).

FIG. 9 includes a table and a graph referring to another example of the present invention and indicating relative changes of a hErih1 gene using RNAi in a renal cortical epithelial cell (HRCE) and a renal epithelial cell (HRE).

FIG. 10 includes a table and a graph referring to another example of the present invention and indicating relative changes of a hErih2 gene using RNAi in a renal cortical epithelial cell (HRCE) and a renal epithelial cell (HRE).

FIG. 11 includes a table and a graph referring to another example of the present invention and indicating relative changes of a Bmal2 gene using RNAi in a renal cortical epithelial cell (HRCE) and a renal epithelial cell (HRE).

FIG. 12 is a graph referring to a still another example of the present invention and indicating changes in hErih genes using RNAi in an ovarian cancer cell strain (KF) sensitive to an anticancer agent and in an ovarian cancer cell strain (KFR) that is isolated from the KF and resistant to an anticancer agent.

### DESCRIPTION OF THE INVENTION

As described above, the novel gene of the present invention comprises any one of the DNAs of (a) to (e) below:
(a) a DNA comprising any one of the base sequences represented by SEQ ID NOS: 1 to 4;
(b) a DNA that comprises a base sequence derived from the base sequence of the DNA (a) by deletion, substitution or addition of one to several bases and encodes a protein interacting with BMAL2 protein;
(c) a DNA that comprises a base sequence derived from the base sequence of the DNA (a) by deletion, substitution or addition of one to several bases and is hybridizable with the DNA (a) under stringent conditions;
(d) a DNA that comprises a base sequence derived from the base sequence of the DNA (a) by deletion, substitution or addition of one to several bases and has a homology of 90% or higher with the DNA (a); and
(e) a DNA comprising a base sequence that is complementary to any one of the DNAs (a) to (d).

In the above DNAs (b)-(d), the number of bases that can be deleted, substituted or added is 1 to 6 with respect to 50 bases, for example. In the above DNA (c), the stringent conditions for hybridization can be the standard conditions in the field, for example. For example, the temperature condition is preferably ±5° C. from the Tm value of the base sequence represented by the respective SEQ ID NOS, more preferably ±2° C., and further preferably ±1° C. A specific example of the conditions is: 5×SSC solution; 10×Denhardt solution; 100 μg/ml salmon sperm DNA; and, hybridization in a 1% SDS at 65° C.; irrigation (twice) in a 0.2×SSC and 1% SDS at 65° C. for 30 minutes; and subsequently irrigation (twice) in a 0.2×SSC and 0.1% SDS at 65° C. for 30 minutes.

The homology in the above DNA (d) is at least 90% for example, preferably at least 95%, and more preferably at least 97.5%.

It is also preferable that the novel gene of the present invention has a base sequence represented by SEQ ID NO: 5. It is also preferable that the novel gene includes a polynucleotide comprising a base sequence that encodes an amino acid represented by SEQ ID NO: 6 or a polynucleotide comprising a base sequence complementary thereto. The amino acid sequence represented by SEQ ID NO: 6 is encoded by the base sequence represented by SEQ ID NO: 5, for example.

The novel gene of the present invention is, for example, a gene derived from a mammal such as a human, a mouse, a rabbit, a dog, a cow and a swine. The genes represented by SEQ ID NOS: 1 and 2 are derived from the human, and the genes represented by SEQ ID NOS: 3 and 4 are derived from the mouse.

Examples of the proteins (ERIH protein) encoded by the novel genes of the present invention include:

Mouse ERIH1 protein: GenBank Accession No. AY957601
Human ERIH1 protein: GenBank Accession No. AY946007
Mouse ERIH2 protein: GenBank Accession No. AY957602
Human ERIH2 protein: GenBank Accession No. AY946008

Next, the primer of the present invention is a primer for amplifying the polynucleotide comprising the novel Erih gene of the present invention or the partial sequence, and the primer comprises the partial sequence of the novel gene of the present invention. It is also possible, with application of the PCR using the primer for example, to detect the existence of the novel hErih gene in the test sample or to obtain genes corresponding to the novel hErih gene of the present invention from various living organisms, particularly from mammals.

The length of the primer is not limited particularly, and can be set to a typical length for a primer, for example, in a range of 10 to 35 bases.

Next, the probe of the present invention is hybridizable with the novel Erih gene of the present invention, and the probe is a polynucleotide comprising either the novel Erih gene of the present invention or the partial sequence. By hybridizing the entire novel Erih genes of the present invention or the partial sequences as the probe with a test sample, the existence of the novel Erih gene of the present invention can be detected. Conditions for the hybridization are the same as mentioned above.

Next, the methods of detecting the novel Erih gene of the present invention include a method of using the primer or the probe of the present invention. The detection method using the primer includes a step of performing PCR by using the primer of the present invention with respect to a test sample, and a step of investigating the existence of the novel Erih gene depending on the existence of a PCR amplified fragment. Meanwhile, the detection method using the probe includes a step of incubating the test sample and the probe of the present invention, and a step of investigating the existence of the novel Erih gene by detecting the hybridization of the test sample and the probe. The assay according to the present invention is characterized in use of the primer and the probe of the present invention, and the specific operation and the condition can correspond to conventionally known PCR methods or any of various hybridization methods.

Next, the diagnostic marker gene of the present invention is used for the diagnosis of the biological rhythm disturbance, and characterized in that the gene is the novel Erih gene of the present invention.

A disease model animal of the present invention is a model animal for a biological rhythm disturbance, and the model animal is either an animal (knockout animal) in which the novel Erih gene of the present invention is inactivated or an animal in which the Erih gene is overexpressed. Though there is no particular limitation on the kind of the animal, the examples include a non-human animal like a mouse (for example, a mammal other than a human). The knockout animal or the like can be prepared by any of the conventionally known methods such as gene-targeting, without any particular limitations.

Next, the antibody of the present invention is a polyclonal antibody or a monoclonal antibody with respect to a protein (ERIH protein) encoded by the novel Erih gene of the present invention or the partial sequence. Examples of the partial sequence include a polypeptide comprising an amino acid sequence represented by SEQ ID NO: 6 and the partial sequence.

There is no substantial limitation on the method of preparing the antibody. For example, the polyclonal antibody and the monoclonal antibody can be prepared by a conventionally known method of producing an antibody by immunization to an animal. There is no particular limitation on the kind of the host animal to be immunized. Mammals other than a human, for example, a rabbit, a rat, a mouse, a goat, a sheep, a horse, a swine, and a guinea pig; and birds such as a chicken, a pigeon, a duck and a quail can be used. Similarly, the method of administering the antibody is not limited particularly, and examples of applicable methods include the intracutaneous administration, the subcutaneous administration, the intraperitoneal administration, the intravenous administration and the intramuscular administration. Preferable methods include the subcutaneous administration, the intraperitoneal administration, and the intravenous administration. A further preferable method is the subcutaneous administration.

For example, for preparing a polyclonal antibody, an antibody (ERIH protein or the peptide) is administered to any of the above-mentioned host animals for immunization. Then, the blood serum, ascites fluid or the like are collected, from which the antibody can be isolated and purified. The monoclonal antibody can be prepared by, for example, fusing a myeloma cell and an antibody-producing cell such as a spleen cell or a lymphoidocyte in the immunized host animal so as to prepare a hybridoma, bleeding the hybridoma, and isolating a hybridoma cell that produces an antibody with specificity.

There is no substantial limitation on the method of purifying the polyclonal antibody or the monoclonal antibody. Purification can be performed by any of conventionally known methods such as the salting-out, the dialysis, the ion-exchange chromatography, the affinity chromatography, and the electrophoresis.

There is no particular limitation on the method of screening the production or the target antibody. Conventionally-known methods such as the radioimmunoassay (RIA) technique and the enzyme immunoassay (EIA) technique can be applied, for example.

Typically, the thus obtained antibody has an immunoglobulin class of IgM or IgG. Also the thus obtained antibody molecules can be used directly as an antibody. Alternatively, an active fragment of the antibody such as Fab, Fab′, and F(ab′)₂ that can be obtained by enzymegenation can be used as an antibody of the present invention.

Next, the siRNA of the present invention is a double strand siRNA targeting the novel Erih gene of the present invention. Though the sequence of the siRNA of the present invention is not limited particularly as long as the target is the novel Erih gene, for example, it is a double strand siRNA of 21 bases comprising 19 base pairs and 2-base 3′ overhang, and the sequences of the 19 base pairs are represented by any of SEQ ID NOS: 26 to 28 of the Sequence Listing.

In the present invention, “siRNA” denotes a RNA molecule of a short strand that can mediate RNAi, and in general, a double strand low-molecular RNA comprising 21 to 27 bases.

Examples of the double strand siRNA of the present invention include a double strand siRNA targeting both the Erih1 gene and the Erih2 gene, a double strand siRNA targeting the Erih1 gene, and a double strand siRNA targeting the Erih2 gene. Each of these siRNA is a double strand siRNA comprising 19 base pairs and 2-base 3′ overhang. An example of sequences of 19 base pairs excepting the overhang in a double strand siRNA targeting both the Erih1 gene and the Erih2 gene is indicated below. These siRNA can be used alone or two or more kinds of siRNA can be used together. In the sequences below, “T” can be recited as “U”.

TABLE 1

| | | SEQ |
|---|---|---|
| | | ID |
| No. | Sequence | NO |
| No. 2 (116) | AGACCAAATTGCTATTAGA | 26 |
| No. 3 (223) | CCAAGTTGTTCAAGCTCTG | 27 |
| No. 4 (236) | GCTCTGCTTACTCAAAAGG | 28 |

In the present invention, the 3′ overhang denotes parts of 2mer of 3′ end protruding from both ends of the 19 base pairs when two RNA strands (whose 19 bases are complementary sequences) anneal from the 5′ end so as to form the double strand. The sequence of the 2-base 3′ overhang is not limited particularly, but the examples include TT (-thymine.thymine), AU (-adenine.uracil), and AG (-adenine.guanine). The overhang parts in the sense strand and the antisense strand of the siRNA can have the same or different base sequences. For example, the overhang of the sense strand can be AG and the overhang of the antisense strand can be AU. Alternatively, the overhang of the sense strand can be AU and the overhang of the antisense strand can be AG.

It should be noted that the sequences of the 2-base 3′ overhang are not limited to this example. An arbitrary natural nucleic-acid bases (adenine, guanine, thymine, cytosine, uracil), and natural/artificial known modified bases can be used in a range not imposing any substantial influences on the RNAi effect. In general, a ribonucleotide can be used for the nucleotide at the 3′ overhang part, but it is not the sole example. A deoxyribonucleotide, a modified ribonucleotide, and any other known nucleotide analogs can be used in a range not imposing substantial influences on the RNAi effect. Furthermore, as required, the double strand siRNA of the present invention can have a 5′ overhang in place of the 3′ overhang.

Other embodiments for the double strand siRNA of the present invention include a double strand siRNA where one or several bases are modified, substituted, added or deleted in the sequences represented by SEQ ID NOS: 26 to 28 of the Sequence Listing, and which induces the RNA interference specific to the novel Erih gene. Here, ‘several’ denotes 2, 3, or 4, for example.

The double strand siRNA of the present invention can be synthesized chemically or enzymatically in vitro; it can be synthesized in vivo, without any particular limitations. Preferably, the double strand siRNA is produced through a chemical synthesis by a conventionally-known method. As for a synthetic double strand siRNA, the concentration can be adjusted in a simple manner, and contamination can be prevented easily, and thus there are advantages from the viewpoint of safety. For example, when producing an overhang double strand siRNA represented by SEQ ID NO: 26, first, an RNA strand prepared by adding an overhang of 2 bases to the 3′ end of the sequence represented by SEQ ID NO: 26, and also an RNA strand prepared by adding an overhang of 2 bases to the 3′ end of a sequence complementary with the sequence represented by SEQ ID NO: 26 are synthesized chemically respectively. Next, the RNA strands are allowed to anneal under a certain condition so as to obtain an overhang double strand siRNA. It is preferable in a practical use that the double strand siRNA is purified suitably by a conventionally known method as required.

A resistance alleviator of the present invention is a medicine for alleviating the cell's resistance to the anticancer agent, and it is characterized in including the double strand siRNA of the present invention. Even for a case of a cancer cell that exhibits resistance to the anticancer agent, the above-mentioned siRNA of the present invention is useful in alleviating the resistance and improving the sensitivity. For this reason, the resistance alleviator including the siRNA of the present invention can be used also in a medical treatment of cells exhibiting resistance to an anticancer agent for example in order to improve the effect of the anticancer agent.

Though the cells to be applied with the agent are not limited particularly, the examples include ovarian cancer cells and the like. Above all, due to the capability of alleviating the resistance to the anticancer agent, the resistance alleviator of the present invention can be used favorably with respect to cells exhibiting resistance to the anticancer agent. There is no particular limitation on the cells. The cells can be human cells, or can be cells of any mammals other than human beings.

### EXAMPLE 1

By a yeast two-hybrid assay, a BMAL2 protein is used as a decoy protein (bait) so as to screen a novel protein (prey) that interacts with the BMAL2 protein, thereby determining the gene sequence of the novel gene to be a partner of the Bmal2 gene.

(1) Screening of Novel Gene (Partial Sequence) Through Yeast Two-Hybrid

In accordance with the assay of two-hybrid system, the following experiment was carried out. First, from a clone of hBmal2 (Invitrogen Clone ID 3445178) purchased from Invitrogen Corporation, a hBmal2 clone was obtained through a PCR cloning. And, using this hBmal2 clone, a BD fusion vector for expressing a fusion protein of a DNA binding protein (BD) and a hBMAL2 protein was prepared. Three kinds of bait insertion fragments for the vector were prepared. Namely, the sequences encode respectively the 1^{st} to the 56^{th} (bait 1), the 193^{rd} to the 283^{rd} (bait 2), and the 125^{th} to 422^{nd} (bait 3) of the amino acids of the hBMAL2 protein (SEQ ID NO: 7). It has been confirmed that the base sequences of the respective bait insertion fragments correspond to the sequences of Invitrogen Clone ID 34451.78, and that the respective baits do not have self-activation abilities so that they are effective for screening. A vector formed by binding cDNA of a human vascular endothelial cell and a GAL4 transcription activation domain (AD) was determined as a selection library vector (AD fusion vector). A transformant of a yeast in which the above-mentioned three kinds of BD fusion vectors were introduced and a transformant of yeast in which the above-mentioned AD fusion vectors were introduced were connected by a filter-mating method, and a positive colony was selected (two-hybrid primary screening). As a result, a positive clone was obtained through a screening using the bait 3. Plasmid was extracted from the thus obtained colony, and the interaction between the bait and prey due to the third promoter-reporter gene system was checked by using a β-gal assay so that the interaction was confirmed. Similarly, the interaction between a bait for a specificity test and the prey was checked by using the β-gal assay so that the high specificity of the prey was confirmed.

Regarding the gene that encodes the prey obtained in the above-mentioned primary screening, a 5′ one-pass sequencing was carried out to find that a fragment of a human novel protein not registered in the GenBank is encoded. The thus determined partial base sequence is represented by SEQ ID NO: 5, and the amino acid sequence encoded by the partial base sequence is represented by SEQ ID NO: 6. A protein specifically interacting with the hBMAL2 protein is called “hERIH protein”, and a gene encoding this protein is called “hErih gene”.

(2) Determination of Entire Sequences of hErih gene Encoding hERIH Protein

The sequences determined in the above paragraph (1) denote sequences of sites of hERIH protein specifically interacts with hBMAL2 protein, and the sites are for reacting with the hBMAL2 protein. Therefore, further the sequences at the 5′ side and the 3′ side of the hErih protein were determined. Moreover, the entire base sequences were determined, and according to the base sequences, the entire amino acid sequences of the hERIH protein were determined. In the determination of the base sequences, first, the conventionally known 5′RACE method and 3′RACE method were used. Further, based on the information of the partial base sequences in the above (1), genes analogous with the hErih gene were retrieved by use of the computer. As the result showed that the gene was analogous partially with a mouse hypothetical protein XP#358333/NP#766072, the full-length sequence of the hErih gene was determined based on the result and by using PCR and the sequence. As a result, two kinds of base sequences (SEQ ID NOS: 1 and 2) including the above-mentioned sequence represented by SEQ ID NO: 5 were determined. The relatively short gene represented by SEQ ID NO: 1 was named as hErih1 (human Erih1)(endo1), and the relatively long gene represented by SEQ ID NO: 2 was named as hErih2 (human Erih2)(endo2). In a comparison of the exon-intron structures of these genes, exon 11 and exon 12 of the hErih2 gene did not exist in the hErih1 gene.

### EXAMPLE 2

Expressions of the hErih1 gene and the hErih2 gene in the respective organs of a human were checked.

First, primers suitable for the realtime PCR represented by SEQ ID NOS: 8 to 13 were prepared regarding the Bmal2 gene (ARANT/Bmal2/Clif NM#020183), the hErih1 gene and the hErih2 gene. By using Clontech total RNA Master Panel II (Clontech), the levels of gene expressions for the respective organs were compared. The kinds of organs were 20 in total: adrenal gland, bone marrow, brain cerebellum, brain (whole), fetal brain, fetal liver, heart, kidney, liver, lung (whole), placenta, prostate, salivary gland, skeletal muscle, testis, thymus, thyroid gland, trachea, uterus, and spinal cord. The standard curve was formed with plasmid, and the template was cDNA1 μl (Total RNA: 50 ng chemical equivalent).

(Primer)
hErih1 gene

SEQ ID NO: 8

Forward primer: 5′-GGGAAGAACCTCAGGAAAAGG-3′

SEQ ID NO: 9

Reverse primer: 5′-CGATCCCACTGTCCAATGTTC-3′
hErih2 gene

SEQ ID NO: 10

Forward primer: 5′-CAGCCCTGTTGCATGTTCA-3′

SEQ ID NO: 11

Reverse primer: 5′-GCCCTGAGAACTGCCCTTTA-3′
Bmal2 gene

SEQ ID NO: 12

Forward primer: 5′-GTGAGCCTGGAGAAGCATCA-3′

SEQ ID NO: 13

Reverse primer: 5′-CCAGCACCAAGTACTGTTCCA-3′

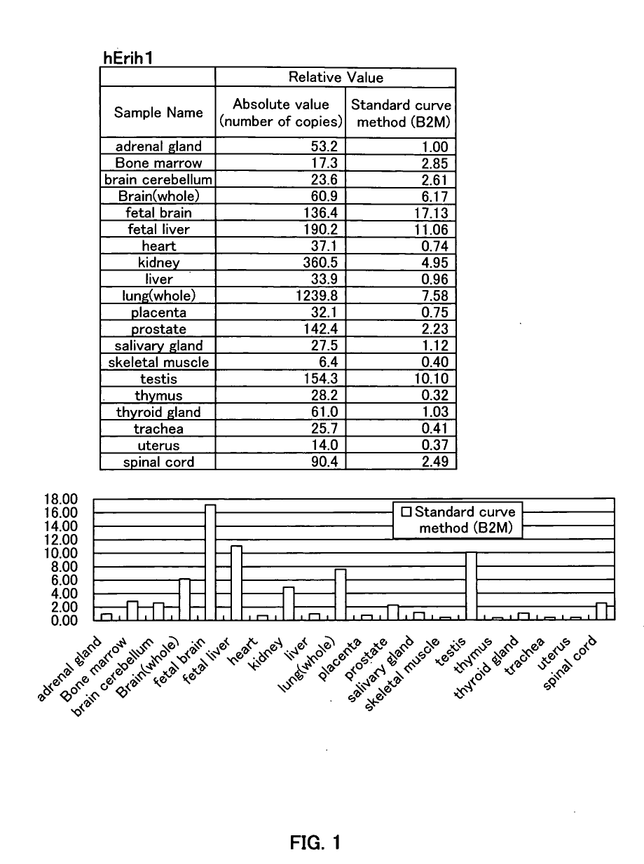

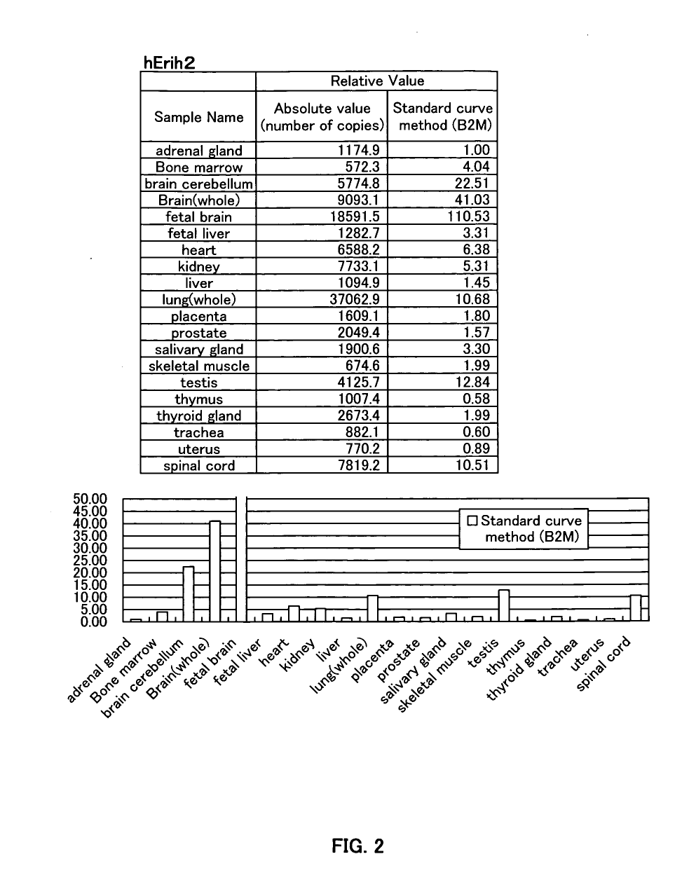

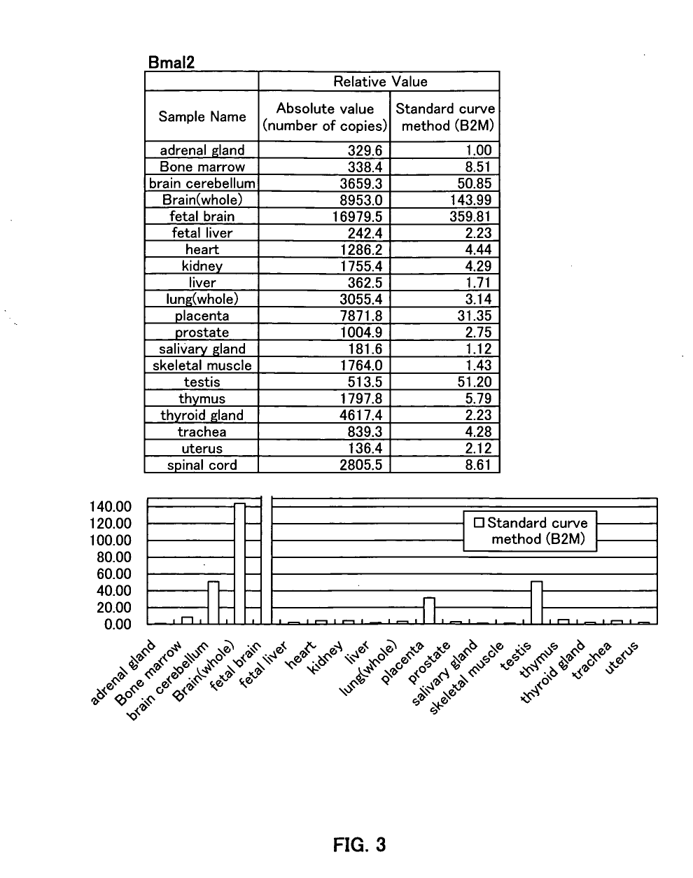

The results are shown in the tables and graphs of FIGS. 1-3. FIG. 1 shows the result for the hErih1 gene, FIG. 2 shows the result for the hErih2 gene, and FIG. 3 shows the result for the Bmal2 gene. The relative values were calculated on the basis of the expression (1.0) of the adrenal gland. As shown in these tables and graphs, a large amount of expression was observed in the brain, the liver and the kidney. For the internal standard gene for expression, Human B2M (beta-2-microglobulin) gene was used. The comparisons in the expression levels after correction are indicated below.

B2M Correction

Bmal2 gene expression: bone marrow<placenta<brain cerebellum<testis<brain (whole)<fetal brain
Erih1 gene expression: kidney<brain (whole)<lung<testis<fetal liver<fetal brain
Erih2 gene expression: bone marrow<lung<testis<brain cerebellum<brain (whole)<fetal brain

### EXAMPLE 3

Since the clock genes are reserved between different species in general, it was checked whether homologues corresponding to the hErih1 gene and the hErih2 gene exist in animals other than the human.

For the mouse, checking was carried out experimentally by a colony plaque hybridization assay and a PCR assay using a primer designed based on XP#358333/NP#766072. With respect to the animals other than the mouse (i.e., the rat, the rabbit, the dog and the monkey), the analogous retrievals were conducted on the NCBI website.

The result shows that, for the mouse, mErih1 (SEQ ID NO: 3) and mErih2 (SEQ ID NO: 4) corresponding to the hErih1 gene and hErih2 gene were found by the PCR. The result of retrieval of these base sequences by use of a computer showed a partial analogy with NM#172484. As a result of comparison between ‘mErih1 gene and mErih2 gene’ and ‘XP#358333 and /NM#172484’, the mErih1 gene and mErih2 gene were regarded as splicing variants. This result implies that the hErih1 gene and hErih2 gene are splicing variants as well.

Concerning the animals other than the mouse, retrieval was conducted on the computer. According to the retrieval result, a gene homologue corresponding to the Erih1 gene and Erih2 gene did not exist for Amphibia, Pisces, and Insecta. In contrast, for Mammalia including the rabbit, the dog, the cow and the swine, the existence of a gene homologue corresponding to the Erih1 gene and Erih2 gene was confirmed.

### EXAMPLE 4

For the purpose of producing antibodies respectively for a hERIH1 protein, a hERIH2 protein, a mERIH1 protein and a mERIH2 protein, peptides to provide an immunity source were prepared.

For obtaining antibodies that distinguish and specifically identify the respective proteins, parts for distinguishing each gene were cloned to synthesize polypeptides. Specifically, the polypeptides were encoded by the common/specific region of the hErih1 gene and the hErih2 gene, the common/specific region of the mErih1 gene and the mErih2 gene, and the common/specific region of the hErih1 gene and the mErih1 gene. Hereinafter, the respective base sequences, amino acid sequences and the estimated molecular weights are recited.

TABLE 2
Base sequence and ammo acid sequence
hErih1 gene-hErih2 gene

| | |
|---|---|
| Common region: | base sequence represented by SEQ ID NO: 14 |
| | amino acid sequence represented by SEQ ID NO: 15 |
| Estimated molecular | 21,351.95 Da (196 amino acids) |
| weight: | |
| Specific region: | hErih2 gene |
| | base sequence represented by SEQ ID NO: 16 |
| | amino acid sequence represented by SEQ ID NO: 17 |
| Estimated molecular | 22,440.39 Da (214 amino acids) |
| weight: | |

mErih1 gene-mErih2 gene

| | |
|---|---|
| Common region: | base sequence represented by SEQ ID NO: 18 |
| | amino acid sequence represented by SEQ ID NO: 19 |
| Estimated molecular | 31,703.20 Da (286 amino acids) |
| weight: | |
| Specific region: | mErih2 gene |
| | base sequence represented by SEQ ID NO: 20 |
| | amino acid sequence represented by SEQ ID NO: 21 |
| Estimated molecular | 24,898.07 Da (222 amino acids) |
| weight: | |

hErih1 gene-mErih1 gene

| | |
|---|---|
| Common region: | base sequence represented by SEQ ID NO: 22 |
| | amino acid sequence represented by SEQ ID NO: 23 |
| Estimated molecular | 23,201.21 Da (210 amino acids) |
| weight: | |
| Specific region: | mErih1 gene |
| | base sequence represented by SEQ ID NO: 24 |
| | amino acid sequence represented by SEQ ID NO: 25 |
| Estimated molecular | 22,379.27 Da (208 amino acids) |
| weight: | |

### EXAMPLE 5

For an analysis of the functions of the hErih genes, an experiment using siRNA was carried out.

The cultivated cells used in this example were derived from the vascular endothelial cell in Example 1 and from the organs (brain, lung and kidney) where more hErih genes were expressed in Example 2. Into the cell culture media, either siRNA regarding the Bmal2 gene or siRNA regarding the hErih gene was added to knock down the Bmal2 gene, the hErih1 gene or the hErih2 gene, and to confirm the changes in the expressions of the Bmal2 gene, the Erih1 gene and the Erih2 gene. At the same time, changes in the expressions of the other genes were checked by a microarray.

Specifically, HPAEC (pulmonary artery endothelial cell) was used for the vascular endothelial cell; an astrocyte was used for the brain; NHBE (bronchial/tracheal epithelial cell) and SAEC (small airway epithelial cell) were used for the lung; and HRCE (renal cortex epithelial cell) and HRE (renal epithelial cell) were used for the kidney. The standard curves were formed with plasmid, and the template was set to cDNA1 μl (Total RNA 50 ng chemical equivalent). For the internal standard gene for expression, Human B2M (beta-2-microglobulin) gene and Human GAPDH were used. The expression levels after correction were calculated respectively.

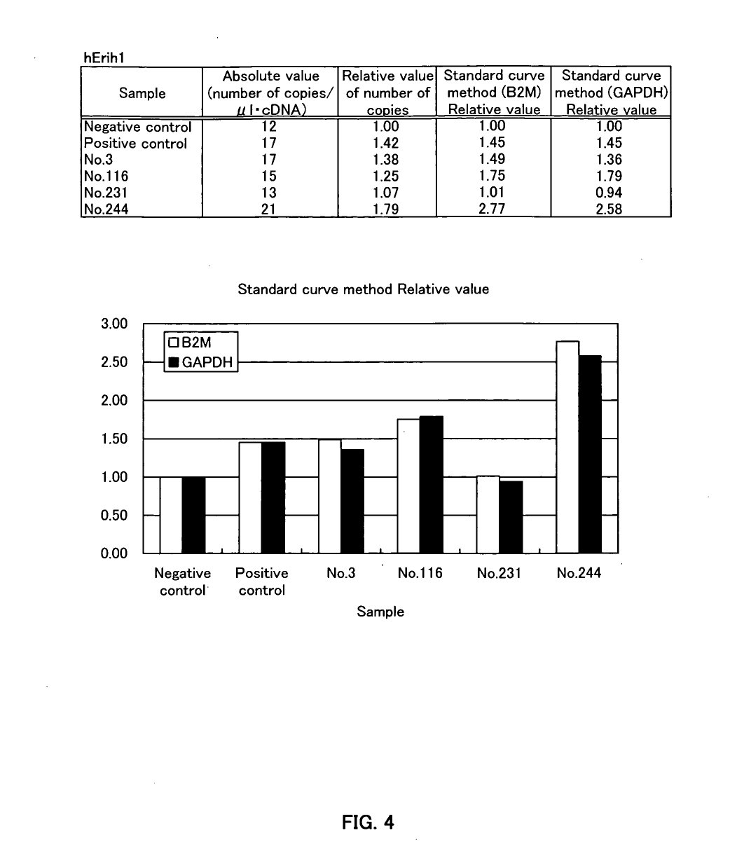

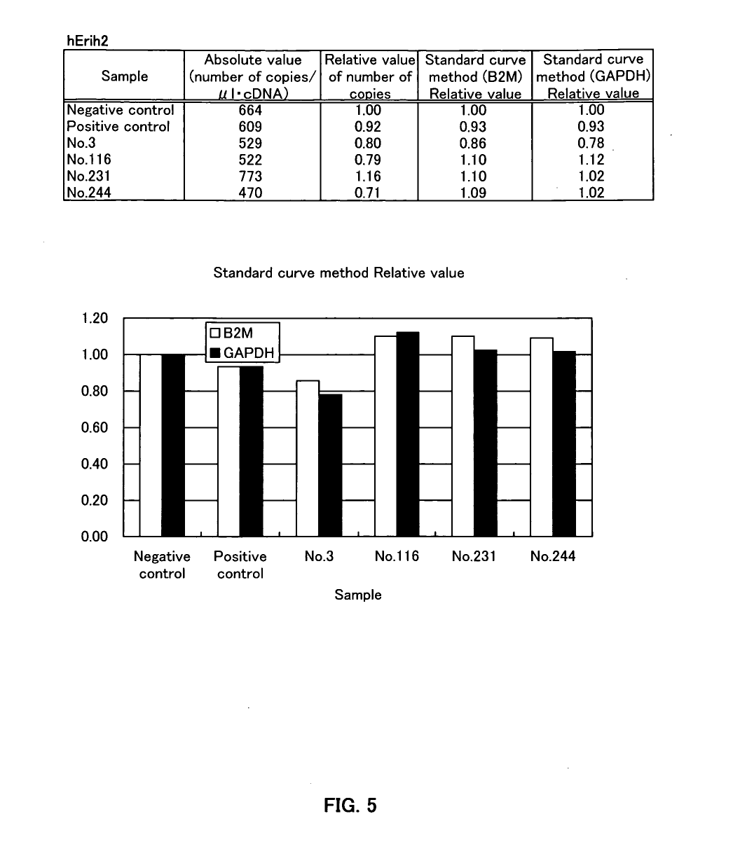

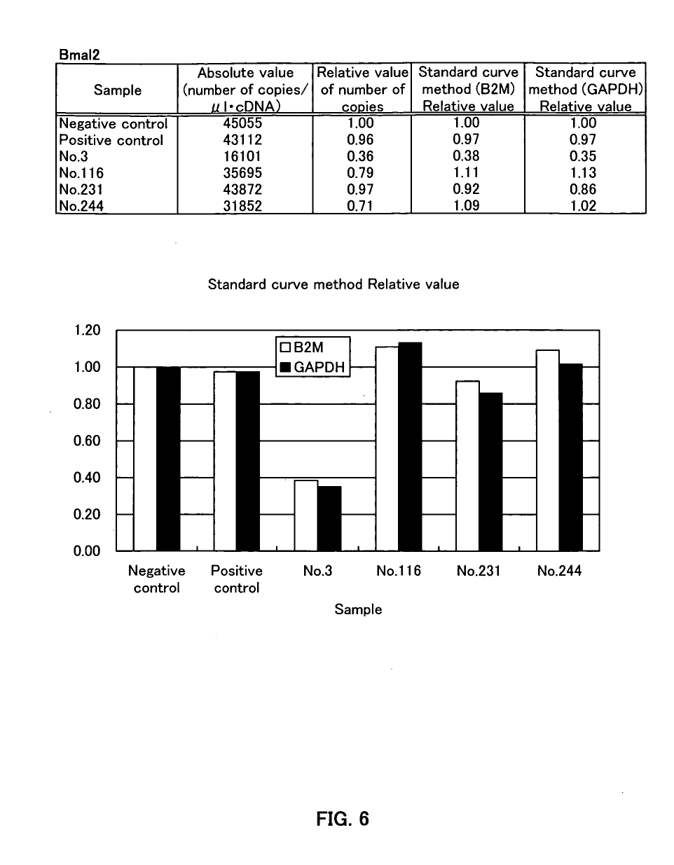

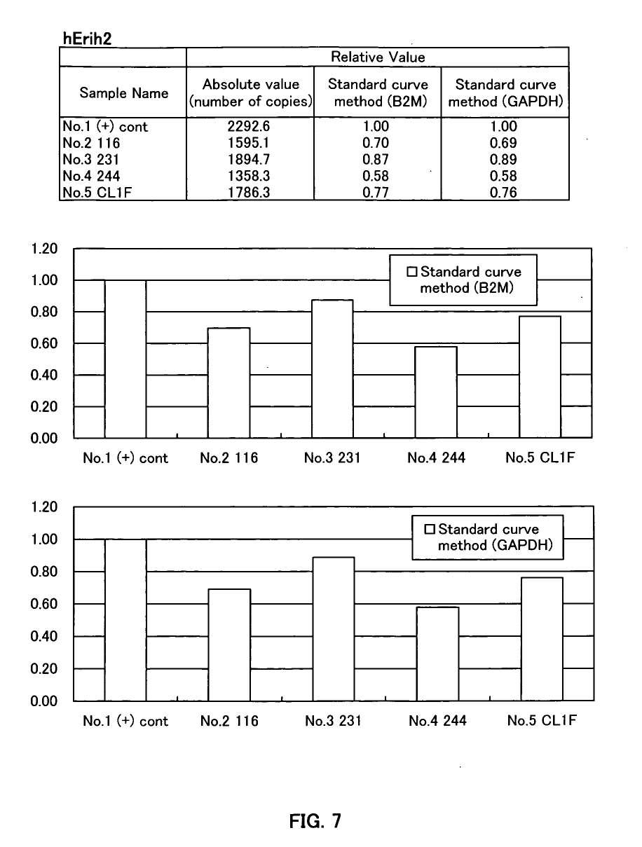

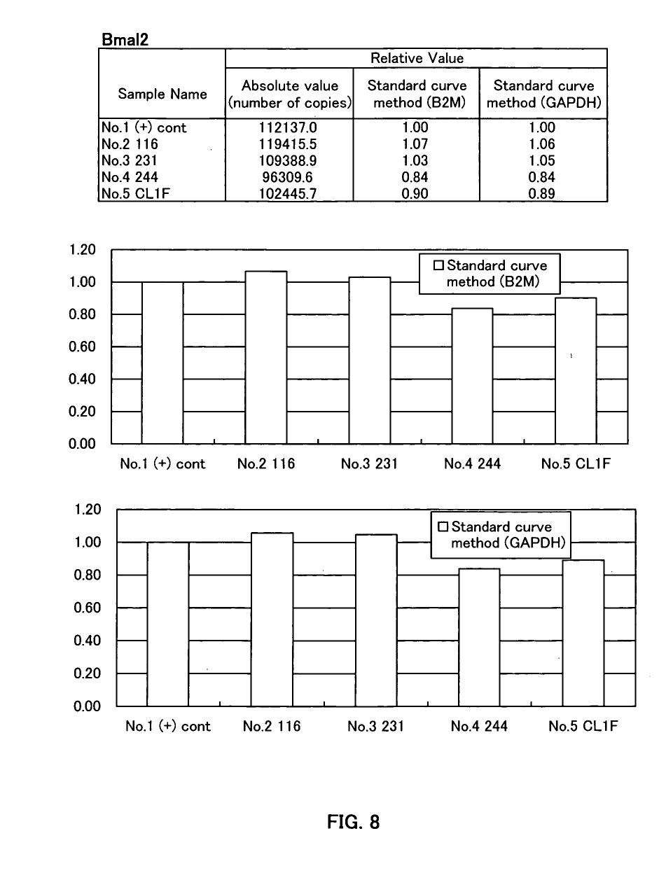

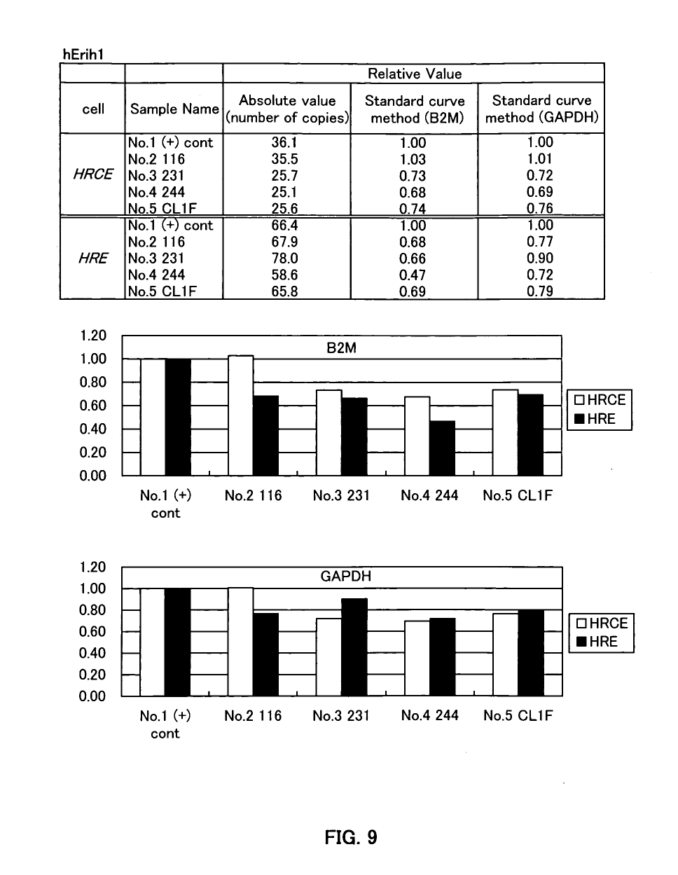

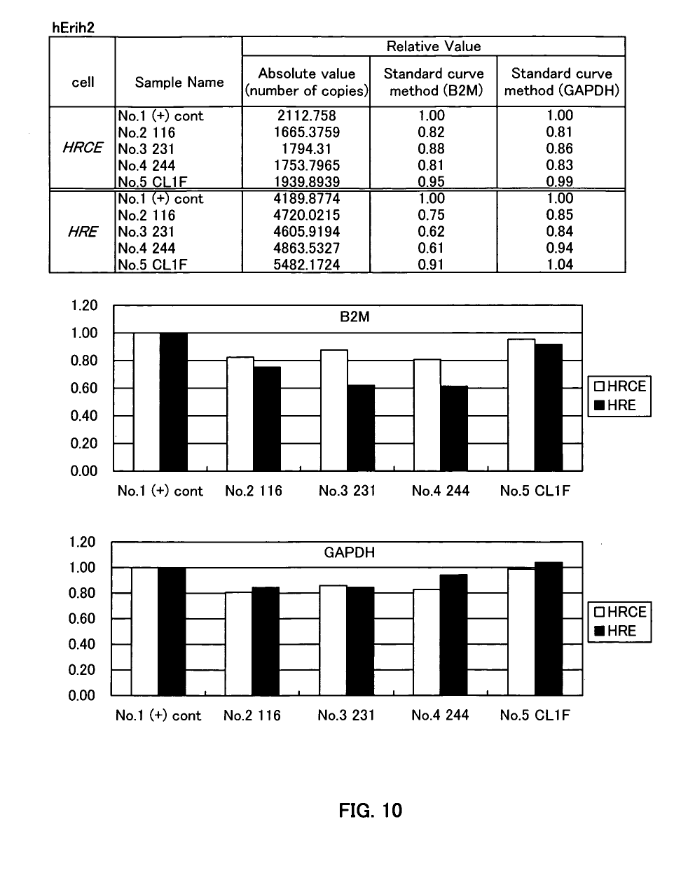

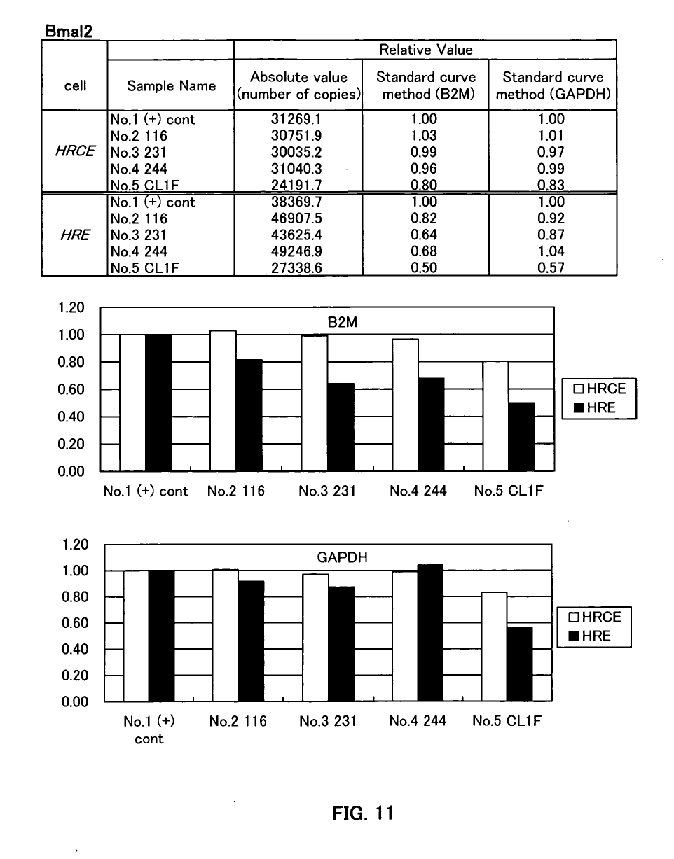

The results for HPAEC are shown in FIGS. 4-6, the results for NHBE are shown in FIGS. 7-8, and the results for HRCE and HRE are shown in FIGS. 9-11 respectively. The controls were not substituted to the knockdown by the siRNA. For the relative values in FIGS. 4-6, the expression of the negative control was set as “1.0” on which the calculation was based. For the relative values in FIGS. 7-11, the expression of the control was set as “1.0”, on which the calculation was based.

The sample numbers in the respective drawings are as follows. All of No. 2 (116:116 siRNA), No. 3 (231:231 siRNA) and No. 4 (244: 244 siRNA) are Erih siRNA, which represent the order of the base that was targeted and knocked down, when counted from the 5′ side, among the site corresponding to the partial sequence found in Example 1. The sites are first-found corresponding to the partial sequences, and thus the sites are bound specifically with the Bmal2 gene, and also common to both the Erih1 gene and the Erih2 gene. Every siRNA shown in the Table 3 below has a 3′ overhang (TT) (in brackets).

TABLE 3

| | |
|---|---|
| Sample | SEQ ID NOS and sequence |

| | | | |
|---|---|---|---|
| No. 1 | Control | | |
| No. 116 or | siRNA of hErih (#1) | SEQ ID NO: 26 | AGACCAAATTGCTATTAGA (TT) |
| No. 2 (116) | | | |
| No. 3 (223) | siRNA of hErih (#2) | SEQ ID NO: 27 | CCAAGTTGTTCAAGCTCTG (TT) |
| No. 4 (236) | siRNA of hErih (#3) | SEQ ID NO: 28 | GCTCTGCTTACTCAAAAGC (TT) |
| No. 5 | siRNA of Bmal2 gene (#3) | SEQ ID NO: 29 | CCAGAGTACTGTTGCTGTC (TT) |
| (CLIF) | CLIF siRNA = ARNT siRNA = | | |
| | Bmal2 siRNA | | |

Here, each of the siRNAs is a double strand siRNA, and the 3′-end 2 bases (TT) of the sense strand and the antisense strand are overhang (in brackets in the above table).

As shown in these tables, for the Bmal gene, the expression tends to decrease only when Bmal2 siRNA (=Arnt siRNA) is added. In contrast, for the hErin1 gene and the hErih2 gene, the expressions tend to decrease not only in the case of adding Bmal2 siRNA (=Arnt siRNA) but also in the case of adding three kinds of Erih siRNA. Namely, when the Bmal2 gene is suppressed by Bmal2 siRNA, both the Erih1 and Erih2 are suppressed. In contrast, when the Erih1 and Erih2 are suppressed by Erih siRNA, the Bmal2 merely is suppressed. This result suggests that the Bmal2 gene is upstream and the Erih genes are downstream.

### EXAMPLE 6

In this example, an anticancer-agent-sensitive ovarian cancer cell strain KF and an anticancer-agent-resistant ovarian cancer cell strain KFR isolated from the KF were used to see whether a change to an anticancer-agent sensitivity is observed as a result of addition of siRNA of the Erih gene.

(Anticancer Agent Solution)

For the anticancer agent, Oxaliplatin (L-OHP) or Diplatin was used, which was mixed in a solvent so as to prepare an L-OHP solution and a Diplatin solution as anticancer agent solutions.

(Assay)

A siRNA transfection to cells was carried out using a RNAi Starter kit of QIAGEN and in accordance with its protocol.

First, on the day before the transfection, the cells were suspended in a medium, and 0.5 ml of the suspension was sprayed on 24 well plates and cultivated in a 5% CO₂ atmosphere.

On the day of the transfection, 1 μg of siRNA was diluted to 100 μl in the medium, in which a reagent RNAiFect Transfection Reagent (6 μl) of the above-mentioned kit was mixed. The mixture solution was incubated at room temperature for 10 to 15 minutes in order to form a transfection complex. Meanwhile, the medium was aspirated from the well of the plates cultivating the cells, and a 300 μl of new medium was added as the substitute. The transfection complex (one drop) obtained by the incubation and also the anticancer agent solution were added to the cells in the well, and the plates were rotated gently to homogenize the distribution of the transfection complex and the anticancer agent solution. And the cells were incubated with the transfection complex in the 5% CO₂ atmosphere. The viabilities of the cells were checked in the following manner.

The below-described siRNAs were used alone or in combination with one or two of the other kinds of siRNAs. When two or more kinds of siRNAs were combined, siRNAs of 1 μg in total were diluted in the medium to 100 μl.

TABLE 4

| | |
|---|---|
| SiRNA | SEQ ID NOS and sequence |

| | | | |
|---|---|---|---|
| B-Mal(2) | siRNA of Bmal2 gene | SEQ ID NO: 29 | CCAGAGTACTGTTGCTGTC (TT) |
| Erih(1) | siRNA of hErih (#1) | SEQ ID NO: 26 | AGACCAAATTGCTATTAGA (TT) |
| Erih(2) | siRNA of hErih (#2) | SEQ ID NO: 27 | CCAAGTTGTTCAAGCTCTG (TT) |
| Erih(3) | siRNA of hErih (#3) | SEQ ID NO: 28 | GCTCTGCTTACTCAAAAGC (TT) |

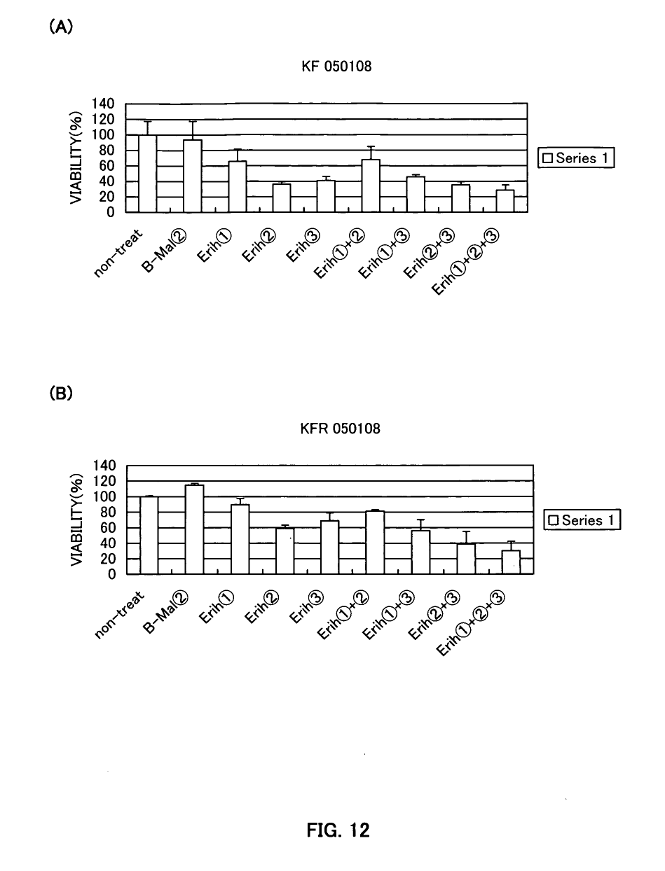

A control (non-treated) was prepared by cultivation in the same manner except that the transfection complex and the anticancer agent solution were not added. The viability (%) was set as a relative percentage when the percentage of the living cells of the control was 100%. The results are shown in FIG. 12. In FIG. 12, the numerical figures in the X-axis denote the numbers of siRNA indicated in the above Table 4. The concentration of the anticancer agent in FIG. 12 denotes the final concentration in the treatment.

FIG. 12 includes graphs for showing the cell viabilities when L-OHP was used as the anticancer agent. FIG. 12(A) shows the result for the sensitive strain KF, and FIG. 12(B) shows the result for the resistant strain KFR. As shown in these graphs, both the anticancer-agent-sensitive strain KF or the anticancer-agent-resistant strain KFR failed to exhibit any significant decrease in the resistance to the anticancer agent. On the contrary, a significant decrease in the resistance to the anticancer agent was observed when RNAi of the Erih gene was added.

### INDUSTRIAL APPLICABILITY

As mentioned above, it has been clarified that the novel Erih gene of the present invention encodes a novel protein that interacts with the BMAL2 protein. Therefore, use of the gene of the present invention and the detection method allow further clarification of the mechanism of the dock gene to which the Bmal2 refers. Furthermore, a diagnosis and an assay of a biological rhythm disturbance can be carried out effectively.

1. A novel gene comprising any one of the following DNAs (a) to (e):
(a) a DNA comprising any one of the base sequences represented by SEQ ID NOS: 1 to 4;
(b) a DNA that comprises a base sequence derived from the base sequence of the DNA (a) by deletion, substitution or addition of one to several bases and encodes a protein interacting with BMAL2 protein;
(c) a DNA that comprises a base sequence derived from the base sequence of the DNA (a) by deletion, substitution or addition of one to several bases and is hybridizable with the DNA (a) under stringent conditions;
(d) a DNA that comprises a base sequence derived from the base sequence of the DNA (a) by deletion, substitution or addition of one to several bases and has a homology of 90% or higher with the DNA (a); and
(e) a DNA comprising a base sequence that is complementary to any one of the DNAs (a) to (d).

2. The novel gene according to claim 1, comprising a polynucleotide comprising a base sequence represented by SEQ ID NO: 5.

3. The novel gene according to claim 1, comprising either a polynucleotide comprising a base sequence encoding an amino acid sequence represented by SEQ ID NO: 6 or a polynucleotide comprising a complementary base sequence.

4. The novel gene according to claim 1, wherein the gene is derived from at least one mammal selected from the group consisting of a human, a mouse, a rabbit, a dog, a cow and a swine.

5. A primer for amplifying a polynucleotide comprising the novel gene according to claim 1 or the partial sequence, the primer being a polynucleotide comprising the partial sequence of the novel gene according to claim 1.

6. The primer according to claim 5, the primer being a polynucleotide comprising a base sequence represented by SEQ ID NO: 5 or a polynucleotide comprising the partial sequence.

7. A method of detecting a novel gene comprising any one of the following DNAs (a) to (e):
(a) a DNA comprising any one of the base sequences represented by SEQ ID NOS: 1 to 4;
(b) a DNA that comprises a base sequence derived from the base sequence of the DNA (a) by deletion, substitution or addition of one to several bases and encodes a protein interacting with BMAL2 protein;
(c) a DNA that comprises a base sequence derived from the base sequence of the DNA (a) by deletion, substitution or addition of one to several bases and is hybridizable with the DNA (a) under stringent conditions;
(d) a DNA that comprises a base sequence derived from the base sequence of the DNA (a) by deletion, substitution or addition of one to several bases and has a homology of 90% or higher with the DNA (a); and
(e) a DNA comprising a base sequence that is complementary to any one of the DNAs (a) to (d);
the method comprising steps of: performing PCR with respect to a test sample by using the primer according to claim 5; and investigating the existence of the novel gene in accordance with the existence of a PCR amplified fragment.

8. A probe hybridizable with the novel gene according to claim 1, the probe being a polynucleotide comprising the novel gene according to claim 1 or the partial sequence.

9. A method of detecting a novel gene comprising any one of the following DNAs (a) to (e):
(a) a DNA comprising any one of the base sequences represented by SEQ ID NOS: 1 to 4;
(b) a DNA that comprises a base sequence derived from the base sequence of the DNA (a) by deletion, substitution or addition of one to several bases and encodes a protein interacting with BMAL2 protein;
(c) a DNA that comprises a base sequence derived from the base sequence of the DNA (a) by deletion, substitution or addition of one to several bases and is hybridizable with the DNA (a) under stringent conditions;
(d) a DNA that comprises a base sequence derived from the base sequence of the DNA (a) by deletion, substitution or addition of one to several bases and has a homology of 90% or higher with the DNA (a); and
(e) a DNA comprising a base sequence that is complementary to any one of the DNAs (a) to (d)
the method comprising steps of: incubating a test sample and the probe according to claim 8; and detecting the hybridization of the test sample and the probe so as to investigate the existence the novel gene.

10. A diagnostic marker gene used for a diagnosis of a biological rhythm disturbance, the gene being the novel gene according to claim 1.

11. A biological rhythm disturbance model animal, wherein the novel gene according to claim 1 is deactivated or overexpressed.

12. A double strand siRNA of 21 bases comprising 19 base pairs and 2-base 3′ overhang, wherein the sequences of the 19 base pairs are any of SEQ ID NOS: 26 to 28 of the Sequence Listing, and the target is the novel gene according to claim 1.

13. A resistance alleviator for alleviating a resistance of a cell to an anticancer agent, the alleviator comprising the double strand siRNA according to claim 12.

14. The resistance alleviator according to claim 13, wherein the cell is an ovarian cancer cell.

15. The resistance alleviator according to claim 13, wherein the cell is derived from a mammal.


SEQUENCE LISTING (29 sequences)

SEQ ID NO: 1 - DNA, 1569 bp, Homo sapiens
atgcatgagaagctgatacatgaactggaagaggagagacacttacgtcttcaaagcgagaagcggttgcaggaggtgaccctagagtctgaacgcaacagaattcagatgcgtagcttgcagcagcagttctccaggatggaagaaacagtacgaaatctattgcagagtcaaggatctccagagcagaaaaaagaagaaactgttaatataatggtctatcaggaaaagctgtcagaggaagagagaaaacataaggaagctttggaagatcttcacatggtggttgatgaggattcgaggagtgaaagcagcagtacagatgagggaaaagaaaagaccaaattgctattagagagattgaaagctctagaggcagagaattcagcgttggctttggagaatgaaaatcaaagggaacaatatgagcgatgtcttgatgaggtagccaaccaagttgttcaagctctgcttactcaaaaggatctaagagaggaatgtgtgaagttgaaaacaagagtgtttgatttggaacagcagaatcgaacactaagcatcctattccaacagcgagtcagacccacttctgatctgctcctccagaaacttcactcacgtctcttggatctttcatctggagatttgctttcagaggtggaaagaaaccgaagtctgacacagtcacgaactgatgctgaggtgcacgaacaccaactaaatacaaaatcggccttgaaatgccctggcttgggggctgtcatccctggtcatctctgtcctcgaaacagctacagtagcagcagtgaactgtctctttcaagcacctgcagcgagtactccagtggctcctcctacacgtggcacgatgggaagaacctcaggaaaagggtaaaagaggcaaatgaaaacttgcaagaggatgaagacgatgcagttgcagattctgtatttcagagccacatcatagaatccaactgccagatgagaacattggacagtgggatcggaacctttccactcccagactcgggaaatcgctcgacaggacgctacctatgccagccagactccccagaggacgctgagcctctcctgcctctccagtcagccctttctgcagtttcttccatgagagcccaaacccttgaacgtgaagtgccttcctccacagacggccagcgccctgcagatagcgccattgttcattccacatccgaccccatcatgaccgccagagggatgaggcctcttcagagccgcctccccaaaccagcttcctcaggaaaagtcagttcccaaaagcagaatgaagcagagccaaggcctcagacatgctcatcattcggatatgctgaagacccaatggcaagccagccgcttccagactgggggagtgaagttgctgccaccgggacccaggacaaggcacccagaatgtgtacgtactctgccagcggtggcagtaatagtgacagtgacctggactatggagataatggttttggagctggaaggggacagttagtgaaagcactgaagagcgctgccccagaaattgagacaacttga

SEQ ID NO: 2 - DNA, 5526 bp, Homo sapiens
atgcatgagaagctgatacatgaactggaagaggagagacacttacgtcttcaaagcgagaagcggttgcaggaggtgaccctagagtctgaacgcaacagaattcagatgcgtagcttgcagcagcagttctccaggatggaagaaacagtacgaaatctattgcagagtcaaggatctccagagcagaaaaaagaagaaactgttaatataatggtctatcaggaaaagctgtcagaggaagagagaaaacataaggaagctttggaagatcttcacatggtggttgatgaggattcgaggagtgaaagcagcagtacagatgagggaaaagaaaagaccaaattgctattagagagattgaaagctctagaggcagagaattcagcgttggctttggagaatgaaaatcaaagggaacaatatgagcgatgtcttgatgaggtagccaaccaagttgttcaagctctgcttactcaaaaggatctaagagaggaatgtgtgaagttgaaaacaagagtgtttgatttggaacagcagaatcgaacactaagcatcctattccaacagcgagtcagacccacttctgatctgctcctccagaaacttcactcacgtctcttggatctttcatctggagatttgctttcagaggtggaaagaaaccgaagtctgacacagtcacgaactgatgctgaggtgcacgaacaccaactaaatacaaaatcggccttgaaatgccctggcttgggggctgtcatccctggtcatctctgtcctcgaaacagctacagtagcagcagtgaactgtctctttcaagcacctgcagcgagtactccagtggctcctcctacacgtggcacgatgggaagaacctcaggaaaaggcaatcatcacaaaactgggataaaaggctaagtattgattcttcgctcccaagtggctttgctagtcctacaaatgaactacctccaactcgtatcaaggaaagccacattttggaagggctaagaaagctacagaagcgaaaagtgttacttgaacccccatcagtgataaccaaatggggttataaagattgcatgaactcgaatgaaggaatatattctcctggaattaaaagtagcagcctcaaggagtatcccccctgcaaaacagctgacctggggagcccctgcaaggaaccccacaagacatttgtttatgatctagattcccacgttgatgcggacgatgacccttccaccttagcattgctccaagcagttccaaaccagagctgcaggccacatggcagtaaattaacccacagtgtttccgacagtctgtttggctgggagacaaacagaaaacactttctggaaggcacatcctcagtttatcccaaggaaaggcctgaaaagctgacaagttgcgccagcagctgtcccttagagatgaagctgtgcccaagtgtgcagacgcctcaggtacagagggagaggggcccacagggccaaggccatggccgcatggctctcaacctccagctttcagacactgatgacaatgaaacgtttgatgagctgcacatagagagcagtgatgagaaaagtccttcagacgtgtcattggctgccgacaccgataagtccgtggagaacctggatgtccttgtggggtttggaaaatctctatgtgggtctcctgaagaggaggaaaaacaagtgcccatcccttcagagactaggccaaagacttttagtttcattaagcagcaaagagttgtaaaaaggacttcttcagaagaatgtgtgactgtgatatttgatgcggaagacggtgagcccattgaattcagctctcaccagactggggttgtcactgttaccagaaatgagatttccatcaattcaactcctgctggacccaaggcagaacatactgagcttttacctcagggaattgcttgtttacagccaagagctgctgcaagagactatactttctttaaaaggtctgaagaggacactgagaaaaacattccaaaagataatgtagataatgttcccagggtgtccactgaatctttcagctccaggacagtgacacaaaatcctcagcagcaaaagctggtcaaaccaacacacaatatatcatgccagagtaattccaggtcttcggcacccatgggcatctatcaaaagcaaaatctgacaaaaatacctcccaggggcaagtcttcacctcagaaatcaaaactaatggagcccgaagccaccacactactcccttcatctggcctggtgactcttgaaaaatcaccagccttagctcctgggaaactctcacgattcatgaagactgagagctcagggcccctctttgaattacgatcagatccacacattccaaaacattccgcccaacttccgcacagctccaggatgcccagcaggagggactgggtccagtgccccaagagtcagactccagggtcacggtcaaggcctgccattgagtctagtgacagtggagagccccccacgagggatgaacactgtggctctgggccggaggcaggggtgaaatccccttcccctccgccccctccaggcaggtccgtctccctgctggccaggcccagctatgactattcaccagcaccttcatccaccaagtccgaaaccagggtccccagtgaaacagcaaggaccccattcaaatccccgctgctgaaaggaatttctgctccagttatttcttctaatccggccacgacagaagtgcagaggaagaaaccttctgtggccttcaaaaagcctatcttcactcaccctatgccctccccagaagcagtcattcaaacccgatgccctgctcatgccccctccagctccttcaccgtaatggctctggggcctccaaaggtctctccgaagagaggtgtccccaaaacctctcctcgccaaacacttgggaccccacaaagggacataggattacagactcccaggatctctccttcaacccatgagccactggaaatgacgtcctccaaaagtgtatctccagggagaaaaggacaattgaatgatagcgcctccacaccccccaagccttccttcttaggggtaaatgagtcaccatcatctcaggtcagcagttcctcatcatcctcatcacccgccaaaagccataacagccctcatggttgtcaaagtgctcatgagaaaggactgaaaactcgccttccagtgggactcaaagtgctcatgaagtctccccagctgctcaggaaaagttccaccgtgccagggaaacatgaaaaagacagtttaaatgaagcctccaaaagttccgtggctgtgaacaagtctaagccagaggactccaagaatccagcaagcatggagatcacagcgggtgaaagaaatgtgaccctaccggattcacaagcacagggcagtttagctgatgggcttcccctggaaacagcactacaagagccattggaaagtagcatccctgggagtgatggaagggatggggtagataatagatccatgagaagatccctttcctccagcaaaccacacctaaaaccagctctgggtatgaatggcgccaaagcccgcagccacagcttcagtacacactcaggagacaagccttctacgccccccatcgaagggtcaggcaaagtccgcactcagatcattaccaataccgccgagagaggcaattctcttacccggcagaactcttccacggaaagctctcccaacaaggccccttctgctcccatgttggagagtctccccagtgttgggaggccctcggggcacccctcctccgggaagggctccctggggagctcaggcagcttcagcagtcagcatgggagcccaagtaagttgcctttgaggatccctccaaagtctgagggactcctcatcccacctggaaaggaagaccagcaggccttcacccagggagagtgccccagtgccaatgtggctgtacttggggaaccaggcagtgaccgccgcagttgcccacccaccccaacagactgccctgaagccctgcagagcccagggaggactcagcatccaagcacttttgaaacaagcagtacatccaagctagaaacttctggaaggcatccagatgcctctgcaaccgcgactgatgctgtgagttcagaagcccccctctcacccacaatcgaagaaaaggtcatgttgtgcattcaggaaaatgtggaaaagggccaagtgcaaacaaagcccacctctgtggaagcaaagcagaagcctgggccttcttttgccagctggtttggttttcggaagagtagacttccagctctgagtagcaggaaaatggacatctccaaaaccaaagtagaaaagaaagatgcaaaagtcttggggtttggaaacagacaactaaaatcagaaagaaaaaaagagaaaaagaagcctgaactacagtgtgagacagaaaatgagcttatcaaggacaccaagtcagcagataatccagatggcggtttacaaagcaaaaataatcggagaacaccacaagacatttacaaccaactgaagattgaaccaaggaatagacacagccctgttgcatgttcaacgaaagacaccttcatgacggaactcttgaacagagttgataagaaagcagctccacagacagaaagtggatcaagtaatgcttcctgcaggaatgtgttaaagggcagttctcagggctcctgtctcatcggcagctctatcagtactcaaggaaaccacaagaaaaacatgaaaatcaaagccgatatggaagtaccaaaagactccctggtaaaagaggcaaatgaaaacttgcaagaggatgaagacgatgcagttgcagattctgtatttcagagccacatcatagaatccaactgccagatgagaacattggacagtgggatcggaacctttccactcccagactcgggaaatcgctcgacaggacgctacctatgccagccagactccccagaggacgctgagcctctcctgcctctccagtcagccctttctgcagtttcttccatgagagcccaaacccttgaacgtgaagtgccttcctccacagacggccagcgccctgcagatagcgccattgttcattccacatccgaccccatcatgaccgccagagggatgaggcctcttcagagccgcctccccaaaccagcttcctcaggaaaagtcagttcccaaaagcagaatgaagcagagccaaggcctcagacatgctcatcattcggatatgctgaagacccaatggcaagccagccgcttccagactgggggagtgaagttgctgccaccgggacccaggacaaggcacccagaatgtgtacgtactctgccagcggtggcagtaatagtgacagtgacctggactatggagataatggttttggagctggaaggggacagttagtgaaagcactgaagagcgctgccccagaaattgagacaacttga

SEQ ID NO: 3 - DNA, 1017 bp, mouse
atgcacgagaagctgatacatgagttggaagaggaacggcacttgcggctacagagtgaggagcggctgcgggaggtgaccctggagtcagagcgcaacaggatccagatgctcggcctgcagcagcagttttccagaatggaagaaactgtccgaagtctgttgcagagccaaggatctccagagcagaaaaaggaagaacctgccaagatcacagcctaccaggaaggactacctgatgaagagagaaaagagcaagcagcactgggtgatctccatgtggtggcagctgatgaggactcaagaagcgagcacagtagtgtggaggagggaaaggaagacagcagactgctcctgaagagactgaaggccctggaggcggagaattcagcactggcactggaaaacgagaatcagagagaacagtatgagcgatgtcttgatgaggtagccaaccaagttgttcgagctctactcactcaaaaggatctgagggaggagtgtgtaaagttgaaaacaagagtgtttgacttggagcaacagaaccgagcactcagcctcctgttccaacagcgagtcagaccagcttctgatgtgcttctccagggggccgaggtcaatgaagatgtacaagaagatgaagaagatagagttgctgacactactctccagagccacatgatagaatccaactgccagatgagaacactggacagcgggattggaacctttcccctcccagactcaggaaaccgctcagtgggacgatacatatgccagcaggactctccagaggacactgatgccctcctgcctctccagccagccgcttccatggcatcctctgccagagcccaaacccttgaccgcgaagtgccttcctccacagacagccagagctccccggagtccatgatcatccattccatatctgaccccagcatgatggccagagagatgagagcctcacagagtcaccttcccaagcctacctcctcaggaaccgaaacaagttga

SEQ ID NO: 4 - DNA, 5334 bp, mouse
atgcacgagaagctgatacatgagttggaagaggaacggcacttgcggctacagagtgaggagcggctgcgggaggtgaccctggagtcagagcgcaacaggatccagatgctcggcctgcagcagcagttttccagaatggaagaaactgtccgaagtctgttgcagagccaaggatctccagagcagaaaaaggaagaacctgccaagatcacagcctaccaggaaggactacctgatgaagagagaaaagagcaagcagcactgggtgatctccatgtggtggcagctgatgaggactcaagaagcgagcacagtagtgtggaggagggaaaggaagacagcagactgctcctgaagagactgaaggccctggaggcggagaattcagcactggcactggaaaacgagaatcagagagaacagtatgagcgatgtcttgatgaggtagccaaccaagttgttcgagctctactcactcaaaaggatctgagggaggagtgtgtaaagttgaaaacaagagtgtttgacttggagcaacagaaccgagcactcagcctcctgttccaacagcgagtcagaccagcttctgatgtgcttctccagcaatcatcgcggatctgggataaaagactaagtattgattcttcgctcccaagtggcttcgccagccctgcagatgaactacctccgacccgtatcaaagaaagccacattctagagggactgaggaagctacagaagcggaaagtgttccttgaatcaccatcattggtgataagcaaatggggttataaagattgcatgaactcgaatgaagggatctattcgccaggaattaaaaccagcagcctcaaggagtgtccgccctgccaaccgtctggcatggggatcccctgccagaatctccatcagacattcatctatgatgcaaattctcatgaggatgcacatgaagaactcccatcctcagccttgccactaacatctccgagccagggctacagaatacagggcagtaaattgacacacagtgtttcagacagtctgtttaactgggagccacataggaagtactttgcagaaggtacagcctcagtatataccagagaaatgcctgaaaggctagctcgttgtgctagccactgccccttggagaggaagctatgtcccagtatacacctgccctgggtgcaaagagacagggacccacacagccaagttcatggccatgtgcctctgaacctgcagctctcagataccgatgacaatgagaccctaaacgagctacacatagagagcagtgatgagagaagcccttcagacctgtccttgactggggacaccgacaagtccatggagaacctagaggtgggattcagaaagtctgaaggtggctctcgggacatgggagaagaagcatgtcccacccacttagagactagcccgaaggcattcagtttcatcaagcaacaaagggttgtcaagaggacctcttcggaagaatgtgtcacggtcatatttgatgcagaaaatggagagccagtggaattcagctctcaccaggctggagtcgtcactgtcaccagggatgaaatttccatcagctcagcccttgctgggccctcagaggaactcactgaactttcacctcaggaaattactcacttccaggcaggagctactgcaagagactgtccttttctgaagagacctgaagaggagactgcgaaaaacattccaaaagatgaaaatcatattctcacagctccgaatgacttcttcagctcccagactataataactcagaattccccaagaccaaagctcgccaaacccacacagggggtagcctggcagagtaattctagacctgttggaaacatgggtgtctacacaaagcaaggtctgacaaaaatcccttccagaggcaagtcttcacctcagaaatccaaacttgtggagtctatgcccaccatgctggtcccctccagtgccctagtgactcctgaaaagacaccagcatcccctatggggaaactctcacaatgtagaaaggcagagagccctggttctctctgtgatgtacagccagaatcacacattccaaaacaccctgcccaggttcctcatatccccaaaatttccaataagaagaactgggttcagtgccccaggagtcagacttcagggatccagtcacggcatctcatggggagaagtgacagtagtgaaatgaggtccaatggctgtgtctctgcgccacagacaagaatgaggtctccctcccctccaacccctccagggagatcagtgtcactcctggttagacccagttatgattacccaccactaccatcacctgccaaacctgaaactcagggccctggagacgctgtaagtccagggttgaaatccccgctgttgaaaggatcctctgctcctcttgtctcttccagtccagtgatgtcagaaatacagaagaaaccatctgtggcctccagaaagtctgccttccctccagctcttccctcaacccaagcggtagtgcacaccagataccctgaacacatagcctccagtccatttgctgtgacgacttcagggcccccaaaagtctctccaaaaagaggcatccctaaacctccccctcaccatgcactaggaactgcccacatggacacagacttacagacacccaagacttgtccttcaagctgtgaactgctagaggtgacatcttgtaaaagcctgtctccagggagaaaaagacagttgagtgacagcactgtgatgccccacaggccttccttctcaggagtgaatgagtccttgacacctcaggttaacagtccatcctcctcttcctcatcctgtagaaaccaagggtcctcacaaggtggccagaactctcccgagaaaggacctaaaactcgcctcccagtaggactcaaagttctcatgaagtctcctcagttgctcaagaaaacttccttggtaccagggaaacaagaaaaggatagtttaaatgaagcatctcgaagctgtgtagcagcaaacacgtctaatcctgaggactccaaagaccctgcaagtctggagacctcaagagatgagagaaccacagccttactaggtgtgagaggtcccgacagtctagctagaggacttgccttagactcaacaatgccagaatcgttagaatgtagcatctccagagatgatggaagagatggggcagacagcagagccataaaaagatccttctcctccagcaagccacacctaaagcctgctctaggcatgaatggagccaaagctcggagtcagagcttcagcatccacttgggagagaagccttccacacccccaacagaagggccaggcaagattcgaactcagattatcaccaacacggctgaaagaggcaactccctcaccagacagaatatctcaatggaagggtccccaagcaagaccccctctgtctcaacatctgataatctgcccaatgctggaaggcccctgggaaaccccttttccaggcagagttctctctgtagcaccggaagttctagcagccagcacggaagcccaagcaagttacccttgaggatgctcacaaggccggaaggactcctcactcctggagtggaagaccaacggctctctactcaggaagagtgtcctgaaagaggtgtacatgagcaatctagcagtgaccaccataggcatccatctcctctttcagactgccctcagttcctgcacgttccagggaggatgcagcgcccaagcaatttggaaacaagtgggatatctaagctagaaacttctggaaggcatccagatgcttcttcaactgggactagtgtcgtgagcccagaagcccccctctcaccctccatcgaagaaaaggtcatgttgtgcattcaggagaatgtggaaaagggccaagtgcagacaaagtctgcctctgtggaagtgaagcccagggcagggccttcgttcaccagctggtttggttttcgaaagagcaggcttccagctctcagtagcaggagaatggaagcttccaagagtaaagtggaaaagaaagacacaaaactgaaatctgagaggaaaaaagaaaagaagaaagctgaagtacaatcaaagatagaaaacgaactgagtagggggaccaagaaggcagatggtcagagtccagacgatggtctgcaaagctcagagagtctgaaagtatcccaagacatttataaccaaatgaagttggagccaaggaatagacccagccctgctgtgtgcccaacaaaagatgccttcatgacagaacttctaaacagagttgataagagagcctcggtacagagagagagtggatcaaagggtgtttcctgcaggagcgtgttagagggcacctcccagggctcctgtttcgccagcggctctgtgagcactcaaggaagtcaaaagaaaaacatcaaaaccaaagtcgatatggaaaaaccaagagaatccttgggggccgaggtcaatgaagatgtacaagaagatgaagaagatagagttgctgacactactctccagagccacatgatagaatccaactgccagatgagaacactggacagcgggattggaacctttcccctcccagactcaggaaaccgctcagtgggacgatacatatgccagcaggactctccagaggacactgatgccctcctgcctctccagccagccgcttccatggcatcctctgccagagcccaaacccttgaccgcgaagtgccttcctccacagacagccagagctccccggagtccatgatcatccattccatatctgaccccagcatgatggccagagagatgagagcctcacagagtcaccttcccaagcctacctcctcaggaaaacctagttttgaaaagcaaaatggagcagagtcaaaggctcacactggcccatcattcgaatatgctgaggatgcagtggcaagtgagcccctttcaggatggaggggtgaagatgctactactgagacccaggaaccgaaacaagttgaagaaacaaaagaagatcctgagaatagattatctgaaatttccctggagtcattcaataaatttagcagcaatactgtgattttattaggaaaagagaagtctccaagtgaggttgaaggacaaaaggaagagaaaggcaaaaagacagaagtgtcttcgagtagctccgaacggcctggggtggaccgtgtggagtctctgagtgactctctgtatgacagtttctcctcttgtgcaagtcaagcatccaacgaggcctag

SEQ ID NO: 5 - DNA, 267 bp, Homo sapiens
gacaaatgcgttctcgaggtggagagaaaacataaggaagctttggaagatcttcacatggtggttgatgaggattcgaggagtgaaagcagcagtacagatgagggaaaagaaaagaccaaattgctattagagagattgaaagctctagaggcagagaattcagcgttggctttggagaatgaaaatcaaagggaacaatatgagcgatgtcttgatgaggtagccaaccaagttgttcaagctctgcttactcaaaaggataag

SEQ ID NO: 6 - protein, 89 aa, Homo sapiens
DKCVLEVERKHKEALEDLHMVVDEDSRSESSSTDEGKEKTKLLLERLKALEAENSALALENENQREQYERCLDEVANQVVQALLTQKDK

SEQ ID NO: 7 - protein, 583 aa, Homo sapiens
EAAAGGKVLREENQCIAPVVSSRVSPGTRPTAMGSFSSHMTEFPRKRKGSDSDPSQEAHSQTEKRRRDKMNNLIEELSAMIPQCNPMARKLDKLTVLRMAVQHLRSLKGLTNSYVGSNYRPSFLQDNELRHLILKTAEGFLFVVGCERGKILFVSKSVSKILNYDQASLTGQSLFDFLHPKDVAKVKEQLSSFDISPREKLIDAKTGLQVHSNLHAGRTRVYSGSRRSFFCRIKSCKISVKEEHGCLPNSKKKEHRKFYTIHCTGYLRSWPPNIVGMEEERNSKKDNSNFTCLVAIGRLQPYIVPQNSGEINVKPTEFITRFAVNGKFVYVDQRATAILGYLPQELLGTSCYEYFHQDDHNNLTDKHKAVLQSKEKILTDSYKFRAKDGSFVTLKSQWFSFTNPWTKELEYIVSVNTLVLGHSEPGEASFLPCSSQSSEESSRQSCMSVPGMSTGTVLGAGSIGTDIANEILDLQRLQSSSYLDDSSPTGLMKDTHTVNCRSMSNKELFPPSPSEMGELEATRQNQSTVAVHSHEPLLSDGAQLDFDALCDNDDTAMAAFMNYLEAEGGLGDPGDFSDIQWTL

SEQ ID NO: 8 - DNA, 21 bp, Artificial
  primer
gggaagaacctcaggaaaagg

SEQ ID NO: 9 - DNA, 21 bp, Artificial
  primer
cgatcccactgtccaatgttc

SEQ ID NO: 10 - DNA, 19 bp, Artificial
  primer
cagccctgttgcatgttca

SEQ ID NO: 11 - DNA, 20 bp, Artificial
  primer
gccctgagaactgcccttta

SEQ ID NO: 12 - DNA, 20 bp, Artificial
  primer
gtgagcctggagaagcatca

SEQ ID NO: 13 - DNA, 21 bp, Artificial
  primer
ccagcaccaagtactgttcca

SEQ ID NO: 14 - DNA, 591 bp, Homo sapiens
atgggcagcagccatcatcatcatcatcacagcagcggcctggtgccgcgcggcagccatatggctagcatgactggtggacagcaaatgggtcgcggatccactcagcatccaagcacttttgaaacaagcagtacatccaagctagaaacttctggaaggcatccagatgcctctgcaaccgcgactgatgctgtgagttcagaagcccccctctcacccacaatcgaagaaaaggtcatgttgtgcattcaggaaaatgtggaaaagggccaagtgcaaacaaagcccacctctgtggaagcaaagcagaagcctgggccttcttttgccagctggtttggttttcggaagagtagacttccagctctgagtagcaggaaaatggacatctccaaaaccaaagtagaaaagaaagatgcaaaagtcttggggtttggaaacagacaactaaaatcagaaagaaaaaaagagaaaaagaagcctgaactacagtgtgagacagaaaatgagcttatcaaggacaccaagtcagcagataatccagatactcgagcaccaccaccaccaccactgagatccggctgctaa

SEQ ID NO: 15 - protein, 196 aa, Homo sapiens
MGSSHHHHHHSSGLVPRGSHMASMTGGQQMGRGSTQHPSTFETSSTSKLETSGRHPDASATATDAVSSEAPLSPTIEEKVMLCIQENVEKGQVQTKPTSVEAKQKPGPSFASWFGFRKSRLPALSSRKMDISKTKVEKKDAKVLGFGNRQLKSERKKEKKKPELQCETENELIKDTKSADNPDTRAPPPPPLRSGC

SEQ ID NO: 16 - DNA, 645 bp, Homo sapiens
atgggcagcagccatcatcatcatcatcacagcagcggcctggtgccgcgcggcagccatatggctagcatgactggtggacagcaaatgggtcgcggatcctttccactcccagactcgggaaatcgctcgacaggacgctacctatgccagccagactccccagaggacgctgagcctctcctgcctctccagtcagccctttctgcagtttcttccatgagagcccaaacccttgaacgtgaagtgccttcctccacagacggccagcgccctgcagatagcgccattgttcattccacatccgaccccatcatgaccgccagagggatgaggcctcttcagagccgcctccccaaaccagcttcctcaggaaaagtcagttcccaaaagcagaatgaagcagagccaaggcctcagacatgctcatcattcggatatgctgaagacccaatggcaagccagccgcttccagactgggggagtgaagttgctgccaccgggacccaggacaaggcacccagaatgtgtacgtactctgccagcggtggcagtaatagtgacagtgacctggactatggagataatggttttggagctggaactcgagcaccaccaccaccaccactgagatccggctgctaa

SEQ ID NO: 17 - protein, 214 aa, Homo sapiens
MGSSHHHHHHSSGLVPRGSHMASMTGGQQMGRGSFPLPDSGNRSTGRYLCQPDSPEDAEPLLPLQSALSAVSSMRAQTLEREVPSSTDGQRPADSAIVHSTSDPIMTARGMRPLQSRLPKPASSGKVSSQKQNEAEPRPQTCSSFGYAEDPMASQPLPDWGSEVAATGTQDKAPRMCTYSASGGSNSDSDLDYGDNGFGAGTRAPPPPPLRSGC

SEQ ID NO: 18 - DNA, 861 bp, mouse
atgggcagcagccatcatcatcatcatcacagcagcggcctggtgccgcgcggcagccatatggctagcatgactggtggacagcaaatgggtcgcggatccggaaaggaagacagcagactgctcctgaagagactgaaggccctggaggcggagaattcagcactggcactggaaaacgagaatcagagagaacagtatgagcgatgtcttgatgaggtagccaaccaagttgttcgagctctactcactcaaaaggatctgagggaggagtgtgtaaagttgaaaacaagagtgtttgacttggagcaacagaaccgagcactcagcctcctgttccaacagcgagtcagaccagcttctgatgtgcttctccaggaacacctccagaatgcaaaatcaggaacccctgccttgaaacactctggctcaggggttgtcatccctgatcacctctgtcctcgcaacagctgcagcagtggcagtgagctatcgctgtcaagcacctgcagcgagtactccagtggctcctcctacacgtggcaagacaggaagctcattgggaaaaggcaatcatcgcggatctgggataaaagactaagtattgattcttcgctcccaagtggcttcgccagccctgcagatgaactacctccgacccgtatcaaagaaagccacattctagagggactgaggaagctacagaagcggaaagtgttccttgaatcaccatcattggtgataagcaaatggggttataaagattgcatgaactcgaatgaagggatctattcgccaggaattaaaaccagcagcctcactcgagcaccaccaccaccaccactgagatccggctgctaa

SEQ ID NO: 19 - protein, 286 aa, mouse
MGSSHHHHHHSSGLVPRGSHMASMTGGQQMGRGSGKEDSRLLLKRLKALEAENSALALENENQREQYERCLDEVANQVVRALLTQKDLREECVKLKTRVFDLEQQNRALSLLFQQRVRPASDVLLQEHLQNAKSGTPALKHSGSGVVIPDHLCPRNSCSSGSELSLSSTCSEYSSGSSYTWQDRKLIGKRQSSRIWDKRLSIDSSLPSGFASPADELPPTRIKESHILEGLRKLQKRKVFLESPSLVISKWGYKDCMNSNEGIYSPGIKTSSLTRAPPPPPLRSGC

SEQ ID NO: 20 - DNA, 669 bp, mouse
atgggcagcagccatcatcatcatcatcacagcagcggcctggtgccgcgcggcagccatatggctagcatgactggtggacagcaaatgggtcgcggatccggaaaggaagacagcagactgctcctgaagagactgaaggccctggaggcggagaattcagcactggcactggaaaacgagaatcagagagaacagtatgagcgatgtcttgatgaggtagccaaccaagttgttcgagctctactcactcaaaaggatctgagggaggagtgtgtaaagttgaaaacaagagtgtttgacttggagcaacagaaccgagcactcagcctcctgttccaacagcgagtcagaccagcttctgatgtgcttctccagcaatcatcgcggatctgggataaaagactaagtattgattcttcgctcccaagtggcttcgccagccctgcagatgaactacctccgacccgtatcaaagaaagccacattctagagggactgaggaagctacagaagcggaaagtgttccttgaatcaccatcattggtgataagcaaatggggttataaagattgcatgaactcgaatgaagggatctattcgccaggaattaaaaccagcagcctcactcgagcaccaccaccaccaccactgagatccggctgctaa

SEQ ID NO: 21 - protein, 222 aa, mouse
MGSSHHHHHHSSGLVPRGSHMASMTGGQQMGRGSGKEDSRLLLKRLKALEAENSALALENENQREQYERCLDEVANQVVRALLTQKDLREECVKLKTRVFDLEQQNRALSLLFQQRVRPASDVLLQQSSRIWDKRLSIDSSLPSGFASPADELPPTRIKESHILEGLRKLQKRKVFLESPSLVISKWGYKDCMNSNEGIYSPGIKTSSLTRAPPPPPLRSGC

SEQ ID NO: 22 - DNA, 633 bp, Homo sapiens
atgggcagcagccatcatcatcatcatcacagcagcggcctggtgccgcgcggcagccatatggctagcatgactggtggacagcaaatgggtcgcggatccctctcaccctccatcgaagaaaaggtcatgttgtgcattcaggagaatgtggaaaagggccaagtgcagacaaagtctgcctctgtggaagtgaagcccagggcagggccttcgttcaccagctggtttggttttcgaaagagcaggcttccagctctcagtagcaggagaatggaagcttccaagagtaaagtggaaaagaaagacacaaaactgaaatctgagaggaaaaaagaaaagaagaaagctgaagtacaatcaaagatagaaaacgaactgagtagggggaccaagaaggcagatggtcagagtccagacgatggtctgcaaagctcagagagtctgaaagtatcccaagacatttataaccaaatgaagttggagccaaggaatagacccagccctgctgtgtgcccaacaaaagatgccttcatgacagaacttctaaacagagttgataagagagcctcggtacagagagagagtggaactcgagcaccaccaccaccaccactgagatccggctgctaa

SEQ ID NO: 23 - protein, 210 aa, Homo sapiens
MGSSHHHHHHSSGLVPRGSHMASMTGGQQMGRGSLSPSIEEKVMLCIQENVEKGQVQTKSASVEVKPRAGPSFTSWFGFRKSRLPALSSRRMEASKSKVEKKDTKLKSERKKEKKKAEVQSKIENELSRGTKKADGQSPDDGLQSSESLKVSQDIYNQMKLEPRNRPSPAVCPTKDAFMTELLNRVDKRASVQRESGTRAPPPPPLRSGC

SEQ ID NO: 24 - DNA, 627 bp, Homo sapiens
atgggcagcagccatcatcatcatcatcacagcagcggcctggtgccgcgcggcagccatatggctagcatgactggtggacagcaaatgggtcgcggatccccttcctccacagacagccagagctccccggagtccatgatcatccattccatatctgaccccagcatgatggccagagagatgagagcctcacagagtcaccttcccaagcctacctcctcaggaaaacctagttttgaaaagcaaaatggagcagagtcaaaggctcacactggcccatcattcgaatatgctgaggatgcagtggcaagtgagcccctttcaggatggaggggtgaagatgctactactgagacccaggaaccgaaacaagttgaagaaacaaaagaagatcctgagaatagattatctgaaatttccctggagtcattcaataaatttagcagcaatactgtgattttattaggaaaagagaagtctccaagtgaggttgaaggacaaaaggaagagaaaggcaaaaagacagaagtgtcttcgagtagctccgaacggcctggggtggaccgcgtggagtctctgagtactcgagcaccaccaccaccaccactgagatccggctgctaa

SEQ ID NO: 25 - protein, 208 aa, Homo sapiens
MGSSHHHHHHSSGLVPRGSHMASMTGGQQMGRGSPSSTDSQSSPESMIIHSISDPSMMAREMRASQSHLPKPTSSGKPSFEKQNGAESKAHTGPSFEYAEDAVASEPLSGWRGEDATTETQEPKQVEETKEDPENRLSEISLESFNKFSSNTVILLGKEKSPSEVEGQKEEKGKKTEVSSSSSERPGVDRVESLSTRAPPPPPLRSGC

SEQ ID NO: 26 - DNA, 19 bp, Artificial
  overhang siRNA
agaccaaattgctattaga

SEQ ID NO: 27 - DNA, 19 bp, Artificial
  overhang siRNA
ccaagttgttcaagctctg

SEQ ID NO: 28 - DNA, 19 bp, Artificial
  overhang siRNA
gctctgcttactcaaaagg

SEQ ID NO: 29 - DNA, 19 bp, Artificial
  overhang siRNA
ccagagtactgttgctgtc